(19) United States
(12) Patent Application Publication
(10) Pub. No.: US 2011/0082370 A1
(43) Pub. Date: Apr. 7, 2011

(54) ENDOSCOPIC FASCIA TUNNELING

(75) Inventors: Richard W. Ducharme, Winston-Salem, NC (US); Vihar C. Surti, Winston-Salem, NC (US); Tyler Evans McLawhorn, Winston-Salem, NC (US)

(73) Assignee: WILSON-COOK MEDICAL INC., Winston-Salem, NC (US)

(21) Appl. No.: 12/896,365

(22) Filed: Oct. 1, 2010

Related U.S. Application Data
(60) Provisional application No. 61/248,142, filed on Oct. 2, 2009.

(51) Int. Cl. A61B 17/32; A61B 8/00; A61B 19/00; A61B 1/06

(52) U.S. Cl. 600/437; 606/108; 128/899; 600/249

(57) ABSTRACT

Medical systems and methods are provided for accessing a bodily cavity, such as the peritoneal cavity or the thoracic cavity, and for supporting diagnoses or procedures within or adjacent to such cavities. According to one embodiment, a method is provided for accessing an internal bodily cavity of patient, the bodily cavity defined by a cavity membrane, the patient having multiple tissue layers adjacent the bodily cavity including at least a skin layer and a fascia layer. An opening is formed through the skin layer and the fascia layer. An elongate medical device is inserted through the opening and between the fascia layer and the cavity membrane to form a tunnel communicating with the opening. Various medical instruments may then be used within the tunnel, and related medical kits and systems are described.

### CROSS-REFERENCE TO RELATED APPLICATIONS

This invention claims the benefit of U.S. Provisional Application Ser. No. 61/248,142 filed on Oct. 2, 2009, entitled “ENDOSCOPIC FASCIA TUNNELING,” the entire contents of which are incorporated herein by reference.

### FIELD OF THE INVENTION

This invention relates to medical devices and methods for accessing a bodily cavity, and more particularly for tunneling through fascia adjacent the bodily cavity for assisting procedures therein.

### BACKGROUND OF THE INVENTION

Access to internal bodily cavities, such as the peritoneal (abdominal) cavity or the thoracic (chest) cavity, can be gained in several ways for diagnosing conditions or performing medical procedures. Historically, there has been open surgery using relatively large incisions, laparoscopy (e.g. a percutaneous approach through smaller openings in the outer skin and related tissue layers), and more recently translumenal procedures (including natural orifice translumenal endoscopic surgeries or NOTES) which use openings in internal bodily structures. For example, culdoscopy was developed over 70 years ago, and involves transvaginally accessing the peritoneal cavity by forming an opening in the cul de sac. This access to the peritoneal cavity allows medical professionals to visually inspect numerous anatomical structures, as well as perform various procedures such as biopsies or other operations, such as tubal ligation. Many transluminal procedures for gaining access to various body cavities using other bodily lumens have also been developed, and with regard to the peritoneal cavity they include trans-vaginal, trans-gastric and trans-colonic. In laparoscopic and translumenal procedures, the area through which medical devices and instruments are inserted in increasingly small, with non-linear access routes that can make the procedures difficult.

### BRIEF SUMMARY OF THE INVENTION

The present invention provides medical systems and methods for accessing a bodily cavity, such as the peritoneal cavity or the thoracic cavity, and for supporting diagnoses or procedures within or adjacent to such cavities. According to one embodiment, a method is provided for accessing an internal bodily cavity of patient, the bodily cavity defined by a cavity membrane, the patient having multiple tissue layers adjacent the bodily cavity including at least a skin layer and a fascia layer. An opening is formed through the skin layer and the fascia layer. An elongate medical device is inserted through the opening and between the fascia layer and the cavity membrane to form a tunnel communicating with the opening. Various medical instruments may then be used within the tunnel.

Accordingly to more detailed aspects, the method further includes grasping an edge of the fascia layer and lifting the edge of the fascia layer. The fascia layer may be separated from the cavity membrane to form the tunnel, and the elongate medical device may be moved distally to increase the size of the tunnel. When the multiple tissue layers includes a second fascia layer, the step of inserting the elongate medical device may include passing the elongate medical device between the fascia layer and the second fascia layer, whereby the tunnel is formed between the fascia layer and the second fascia layer. Preferably the elongate medical device is an endoscope. The medical instrument may be an ultrasound detector formed as part of the flexible endoscope, or a plurality of magnets (e.g. to be used in conjunction with an attracting magnet to mechanically for a pneumoperitoneum), lighting devices, shielding layers, or various other instruments. In one embodiment, the opening is formed posteriorly of a rib cage of the patient, and the tunnel extends anteriorly from the opening to a position proximate the thoracic cavity.

Additional embodiments, in accordance with the teachings of the present invention, provide a medical kit for accessing an internal bodily cavity of patient The bodily cavity is defined by a cavity membrane, and the patient has multiple tissue layers adjacent the bodily cavity including at least a skin layer and a fascia layer. The medical kit includes a cutting instrument, a medical instrument, and a flexible elongate medical device. The cutting instrument is suitable for forming an opening in the tissue layers. The medical instrument is sized for insertion into the opening, and is capable of engaging and separating tissue layers to form a tunnel entrance. The flexible elongate medical device is sized for insertion into the opening and tunnel entrance and between tissue layers, the flexible elongate device having sufficient rigidity to further separate tissue layers through distal advancement of the elongate medical device through the tunnel entrance to increase the size of the tunnel. According to more detailed aspects, the medical kit may further include a second medical instrument sized for placement within the tunnel and between tissue layers. In one embodiment, the second medical instrument is a plurality of magnets, and preferably the medical kit also includes an actuating magnet for attracting the plurality of magnets and maintaining a space between the tissue layers. In other embodiments, the second medical instrument may be one or more lighting devices or a layer of shielding material, and the kit may include a laparoscopic port sized to be received within the opening.

Another embodiment of the present invention provides a medical system for mechanically positioning tissue layers adjacent a bodily cavity of a patient. The medical system includes a plurality of first magnets, a second magnet, and a frame. The plurality of first magnets are structured to be passed through an opening in the tissue layers and into a tunnel formed between the tissue layers. The second magnet is adapted to be positioned outside of the patient. The frame is structured for connection to the second magnet, and to be supported on at least one of an operating table, a floor, and the exterior of the patient. The second magnet attracts the plurality of first magnets with a collective attractive force sufficient to move and maintain the tissue layers away from the bodily cavity. Preferably, the first magnets each have a smallest cross-sectional area less than about 15 square millimeters.

### BRIEF DESCRIPTION OF THE DRAWINGS

The accompanying drawings incorporated in and forming a part of the specification illustrate several aspects of the present invention, and together with the description serve to explain the principles of the invention. In the drawings:

FIG. 1 is a schematic depiction of a cross-section of an abdomen of a patient;

FIGS. 2-4 depict an enlarged cross-sectional view, partially cut away, of a portion of the patient depicted in FIG. 1;

FIG. 5 is an enlarged cross-sectional view, partially cut away, depicting an embodiment of a medical system deployed within the bodily tissues depicted in FIGS. 2-4;

FIG. 6 is an enlarged cross-section view, partially cut away, depicting an alternate embodiment of a medical system deployed within the bodily tissues depicted in FIGS. 2-4;

FIG. 7 is an enlarged cross-section view, partially cut away, depicting another alternate embodiment of a medical system deployed within the bodily tissues depicted in FIGS. 2-4;

FIG. 8 is a cross-sectional view, partially cut away, depicting an alternate medical device for use with the medical systems of FIGS. 1-7; and

FIG. 9 is a cross-sectional view, partially cut away, depicting an alternate medical device for use with the medical systems of FIGS. 1-7.

### DETAILED DESCRIPTION OF THE INVENTION

The terms “proximal” and “distal” as used herein are intended to have a reference point relative to the user. Specifically, throughout the specification, the terms “distal” and “distally” shall denote a position, direction, or orientation that is generally away from the user, and the terms “proximal” and “proximally” shall denote a position, direction, or orientation that is generally towards the user.

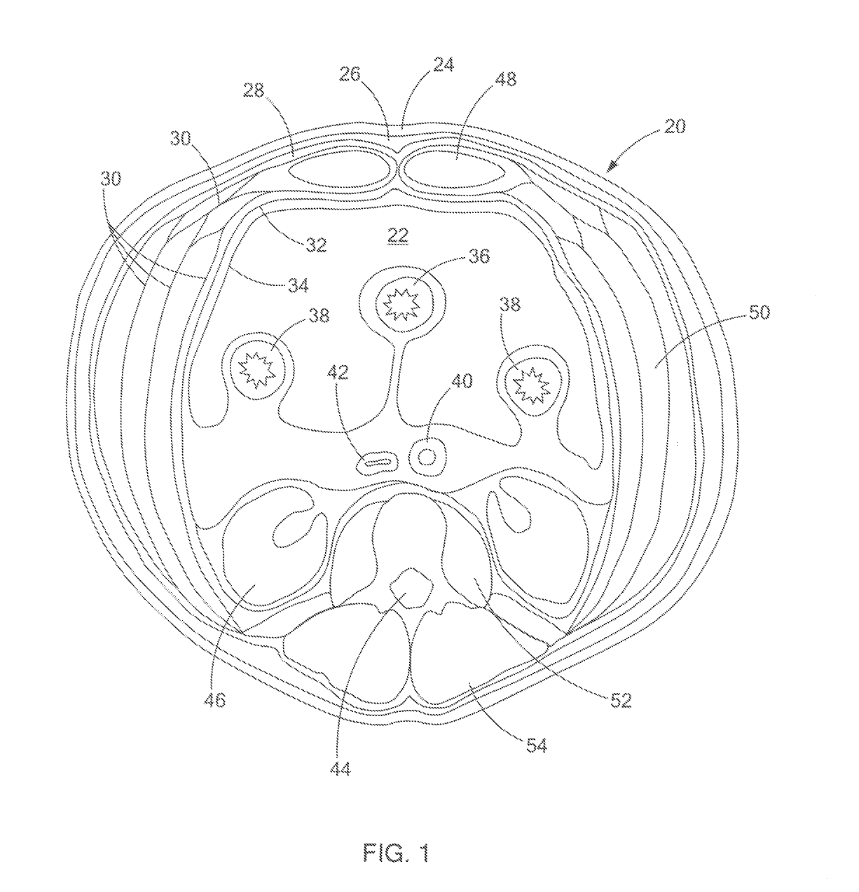

Turning now to the figures, FIG. 1 schematically depicts a cross-sectional view through a patient 20, and in particular through an abdominal region of the patient to show the peritoneal cavity 22. Generally, the patient 20 has an outer skin layer (or layers) 24, an epidural fascia 26 (also known as fatty “Campers” fascia), beneath which lies a superficial fascia 28 (a.k.a. membranous “Scarpas” fascia). Beneath the superficial fascia 28 lies one or more layers of deep fascia 30. Next lies the subserus fascia 32 (a.k.a. transversalis fascia), which lies adjacent the peritoneal membrane 34. The peritoneal cavity 22 is generally defined by the peritoneal membrane 34. It will be recognized that other tissue layers may be present, for example layers of fat may be present between the layers of skin, fascia and membranes. Likewise different areas of the body, and different patients, may include different tissue layers than those described herein. It will likewise be recognized that many areas of the body included multiple tissue layers, and namely multiple fascia layers and cavity membranes.

For reference purposes, other body portions have been identified in FIG. 1, including the large intestine 36, the small intestine 38, the aorta 40, the inferior vena cava 42, the vertebrae or spine 44, the kidneys 46, the rectus abdomenus muscle 48, the oblique muscles 50, the psoas muscle 52 and the back muscles 54. Additional tissue layers and fat may be found between these structures and other tissue layers.

As will be described in more detail herein, methods of accessing the peritoneal cavity 22 will generally include forming an opening through the adjacent tissue layers and forming a tunnel between the tissue layers through which an a medical device(s) may be passed, e.g. a flexible scope or wire guide. Generally, the tissue layers are both strong while being easily separated, especially the fascia layers. Likewise these tissue layers, and especially the fascia layers, are generally transparent or translucent and therefore permit light to pass therethrough. Accordingly, various medical instruments may be utilized within the tunnel, including illumination, guiding, and visualization instruments, as will be described in further detail herein.

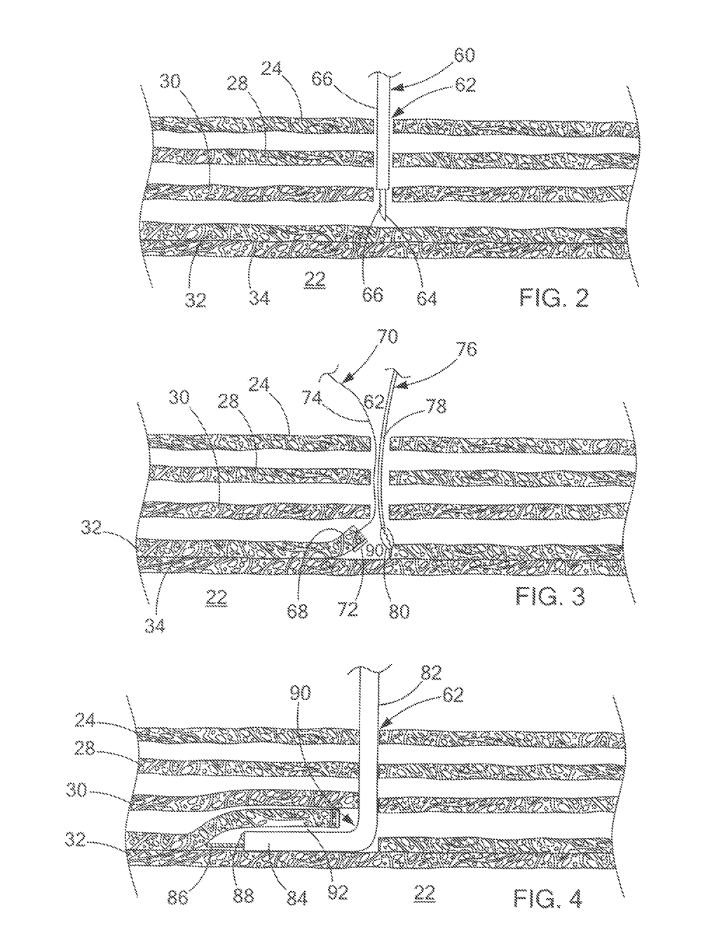

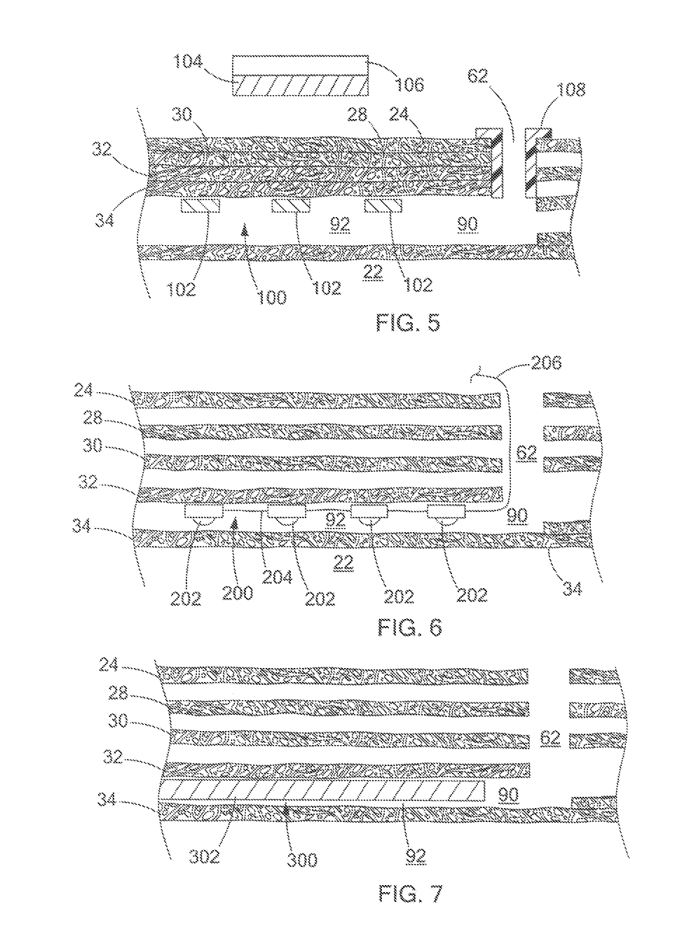

Turning now to FIGS. 2-4, a method for accessing an internal bodily cavity, such as the peritoneal cavity 22 shown in FIG. 1, will now be described. As shown in FIG. 2, a cutting instrument 60, e.g. a mechanical or electro-surgical cutting instrument, is utilized to form an opening 62 through the various layers of tissue, namely the skin 24, superficial fascia 28, and deep fascia 30. The cutting instrument 60 has been depicted as including a trocar 64 having a protective sheath 66, although a scalpel or electro-surgical needle knife can also be used, among other cutting instruments. Preferably the trocar 64 includes one or more markers 66 to accurately identify the location of the distal end of the trocar 64, so that only the desired tissue layers can be cut, as will be described further below. Various imaging or monitoring techniques may also be used to control the depth of the opening 62, including fluoroscopy, ultrasound or direct visualization may be utilized, as will be understood by those skilled in the art. In this manner, the cutting instrument 60 can be controlled to cut through the various tissue layers up to, but not including, the peritoneal membrane 34, although some fascia layers may also be left uncut. Depending upon the particular cutting tool 60 and the particular medical procedure desired to be performed (and other related factors), the opening 62 may be enlarged or otherwise manipulated, e.g. using a dilation balloon or the like. Similarly, a laparoscopic port or other porting device may be employed through the opening 62 (see, e.g., port 108 shown in FIG. 5).

As shown in FIG. 3, an edge 68 of the subserus fascia 32 is engaged and lifted away from the peritoneal membrane 34. This may be accomplished using various instruments, for example, a tissue anchor 70 which has a cross bar 72 connected to a suture 74. The edge 68 of the subserus fascia 32 can be pierced with a delivery needle to position the cross bar 72 on the distal side of the fascia 32, and the suture retracted proximally to lift the edge 68. Proximate the edge 68 of the subserus fascia 32, an entrance 90 is formed for towing between layers of the tissue, here the super subserus fascia 32 and the peritoneal membrane 34. Many other medical instruments may be used to facilitate separating the subserus fascia 32 from the peritoneal membrane 34, including a grasper 76 having an elongated control member 78 and grasping jaws 80, and shown engaging the edge of the fascia 32 opposite the edge 68 in FIG. 3 for illustration purposes. Various instruments may be used, including graspers, barbs, clamps, tweezers and the like. At the same time, the fascia layers and membranes separate relatively easily, and thus no instrument may be used (aside from the accessing device such as the endoscope 82 (FIG. 4)).

Turning now to FIG. 4, the method further includes inserting a flexible endoscope 82 through the opening 62 formed in the tissue layers. It will be recognized by those skilled in the art that many flexible elongate medical devices may be utilized with the methods of the present invention, including other flexible scopes, catheters and steering catheters, wire guides and the like. The endoscope 82 (or other flexible elongate medical device) may be provided as a kit with a tissue cutting device 60 and a tissue engaging device 70/76, using appropriate collective packaging. As shown, the endoscope 82 has been inserted and moved distally through the opening 62, into the entrance 90 between the subserus fascia 32 and peritoneal membrane 34. The endoscope 82 is further moved distally therethrough to create a tunnel 92 between the two tissue layers (e.g. the subserous fascia 32 and peritoneal membrane 34 as shown). Through use of the endoscope 82, formation of the tunnel 92 may also be visualized using the scope's optics 88, as is known in the art.

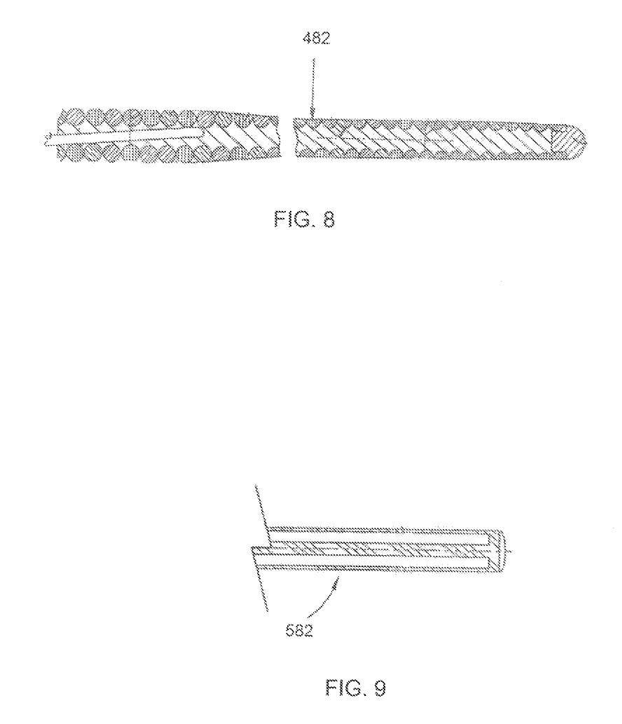

As an endoscope typically includes one or more working channels, a wire guide 86 may be utilized through a working channel and beyond the distal end 84 of the endoscope 82 to facilitate both separation of the tissue layers 32, 34, as well as guide the endoscope 82 through a desired path. It will be recognized that use of the wire guide 86 is not necessary, while at the same time other devices may be employed in conjunction with the endoscope 82 including catheters, e.g. a balloon catheter could be employed to further facilitate formation of the tunnel 92 by repeatedly inflating and deflating the balloon in front of the endoscope 82. Often such additional devices will not be necessary as fascia layers and membranes are generally separated with minimal difficulty. It will also be recognized that a wire guide, catheter or other elongate device may be used alone and without the endoscope 82. Other preferred devices for forming the tunnel 92 include a steerable wire guide 482, such as shown in FIG. 8 and further disclosed in U.S. Pat. No. 6,805,676, and a steerable catheter 582, such as shown in FIG. 9 and further disclosed in U.S. Pat. No. 7,608,056, the disclosures of all of the foregoing patents incorporated herein by reference in their entirety. The steerable wire guide 482 or catheter 582 can also be made as part of the medical kit described above. Supplemental medical instruments and devices, such as those disclosed below, can be delivered over/through the wire guide 482 or catheter 582 and into the tunnel 92, as discussed further hereinbelow.

While FIGS. 2-4 have depicted separating the subserus fascia 32 from the peritoneal membrane 34, it will be recognized that the methods of the present invention may include going between other tissue layers, for example between the deep fascia 30 and the subserus fascia 32, and likewise between many other tissue layers, and especially fascia.

Turning now to FIG. 5, one embodiment of a medical instrument 100 has been shown as deployed between the subserus fascia 32 and the peritoneal membrane 34. In this embodiment, a plurality of first magnets 102 have been shown as spaced along the tunnel 92. A second, attracting magnet 104 has been shown located outside of the body (i.e. proximal to the skin 24). The second magnet 104 has been attached to a frame 106, which in turn can be attached to at least one of an operating table, a floor, and the exterior of the patient 20. Suitable frames 106 or similar support structures are generally known to those skilled in the art. Accordingly, it will be recognized that the medical instrument 100 can be utilized to mechanically create a “pneumoperitoneum”, by virtue of the attracting force between the plurality of magnets 102 and the second magnet 104. The attracting force can be selected based on the particular patient and the anticipated distance between the first and second magnets 102, 104. Preferably, the plurality of magnets 102 have a smallest cross-sectional area less than about 15 square mm so that they may be easily passed through the opening 62 and the tissue layers and the tunnel 92, including through a laparoscopic port 108 which has been inserted into the opening 62 to protect the edges of the adjacent tissue layers. For example, the endoscope 82 may be utilized to deliver the plurality of magnets 102 within the tunnel 92.

In this manner, numerous problems associated with creating a pneumoperitoneum are avoided, such as compartment syndrome, (i.e. air pressure preventing natural blood flow in certain areas) with reduced pain to the patient. The plurality of magnets 102 may be temporally placed between the tissue layers, and then later removed again using a medical device such as the endoscope 82 or other grasping devices such as the device 76 depicted in FIG. 3. Likewise a perfect seal does not need to be maintained on the laparoscopic port 108 since the pneumo does not need to be regulated by a gas machine. The magnets 102 may likewise be connected via a suture or string, and therefore simply pulled out of the tunnel 92 via the suture exiting through the opening 62 and the laparoscopic port 108. It will be recognized that various other devices may be used in place of the plurality of magnets 102, including a balloon filled with magnetic fluid, metal plates (magnetically attractive) or other magnetizable devices.

Turning now to FIG. 6, another embodiment of a medical instrument 200 for use within the tunnel 92 has been depicted. In this embodiment of the medical instrument 200, a plurality of lighting devices 202 have been shown positioned within the tunnel 92. The lighting devices 202 may be LEDs or other lighting devices such a fiber optics. A wire 204 preferably extends along and between each of the plurality of lighting devices 22 and a proximal portion 206 of the wire 204 extends out through the opening 62 in the tissue layers. As previously mentioned, the peritoneal membrane 34 is fairly translucent, and therefore the light emitted from the lighting device 202 may be readily used to illuminate the interior of the peritoneal cavity 22 and facilitate procedures therein.

In FIG. 7, yet another alternate embodiment of a medical instrument 300 has been depicted for use within the tunnel 92. In this embodiment, the medical instrument 300 comprises a sheet 32 formed of shielding material, which can be utilized to protect against various forms of radiation that may be directed towards the peritoneal cavity 22. The sheet 302 may be folded or rolled up and inserted through the opening 62 in the tissue layers and entrance 90 and into the tunnel 92, and then unfolded or unrolled, for example using forceps or other manipulatable devices.

In addition to the medical instruments 100, 200, 300 described above, various other medical instruments may be utilized within the tunnel 92 as will be recognized by those skilled in the art. For example, various device actuators, such as magnetic actuators, may be placed within the tunnel 92 for controlling or otherwise actuating devices within the peritoneal cavity 22. Magnetically actuatable devices often suffer from difficulties with obese patients, e.g. when the magnets do not have sufficient strength to work through large layers of fat. Accordingly, only the peritoneal membrane 34 separates a magnetically actuator in the tunnel 92 (or possibly other tissue layers when the tunnel 92 is formed between, e.g., the fascia layers 26, 28, 30) and therefore much of the fat located on the outer layers of the stomach are bypassed. In essence, the methods of the present invention allow the distance over which the magnetically controlled devices work to be standardized across patients. Likewise multiple medical instruments may be utilized through the opening and within the tunnel 92, for example the lighting device 200 may be utilized in conjunction with such magnetizable controls or the magneto-peritoneum device 100 depicted in FIG. 5.

As one example, a NOTES procedure may be utilized to access the peritoneal cavity 22, either via the mouth and forming an opening in the stomach, via the anus and forming an opening in the colon or intestines, or via the vagina and forming an opening in the cul de sac. Accordingly, the medical devices 100, 200, 300 may be utilized to enhance and facilitate these procedures. Similarly, the same opening 62 (and/or laparoscopic port 108) may be utilized by a medical device which may pierce through the peritoneal membrane 34, such as in typically laparoscopic procedures. In these cases, the medical instruments will already be placed within the tunnel 92, therefore leaving the full area of the opening 62 and port 108 available for use with such other medical devices, such as a laparoscope or laparoscopic instrument.

It will also be recognized that the endoscope 82 depicted in FIG. 4 may have ultrasonic capabilities, or likewise another medical device may be utilized within the tunnel 92 having ultrasound imaging capabilities. As noted above, this method allows bypassing many layers of tissue and fat, and improves the ability to visualize the peritoneal cavity 22 and various structures therein at high resolution. Not only can imaging be completed, but also therapies and biopsies commonly done with an endoscope in the gastro intestinal tract may be performed. For example, a biopsy of the liver or kidney 46 may be taken with an endoscopic ultrasound needle that may be deployed through the endoscope 82 and the tunnel 92, and then pierced through the peritoneal membrane 34 in an appropriate location.

It will also be recognized by those skilled in the art that the methods described herein may be utilized to access other bodily cavities. This includes not only directly forming an opening in the tissue layers above such cavities, but likewise includes tunneling through the body from adjacent one cavity to another cavity within the body. For example, utilizing the opening 62 and tunnel 92 formed in the abdomen, the tunnel 92 between the subserus fascia 32 and membrane 34 may be extended towards the anterior of the patient until the thoracic cavity is accessed. That is, the opening 62 may be formed posteriorly of a rib cage of the patient (e.g. in the navel), and the tunnel 92 may extend anteriorly from the opening to a position proximate the thoracic cavity. In this manner, access to the thoracic cavity is obtained less invasively since the ribs do not need to be broken or separated to gain access thereto. Similarly, a majority of spinal diseases are found on the ventral side of the spine and vertebrae 44. Thus typical percutaneous spinal surgery is cumbersome and not from an ideal mechanical angle. In fact, many cervical spinal surgeries are completed through the ventral portion of the neck to access the back of the neck. Accordingly, the methods described herein allow accessing a deeper tissue layer in the abdomen, and tunneling through the tissue layers allows a scope and/or other medical instrument to gain access to the thoracic cavity and related structures. The diaphragm is also avoided by forming the tunnel between the fascia layers and/or membranes.

It will also be recognized by those skilled in the art that, while the methods described above generally include placing the medical devices and systems through bodily tissue, it will be recognized that the systems, devices and methods may be used on any layer of material (e.g. fabrics, cloth, polymers, elastomers, plastics and rubber) that may or may not be associated with a human or animal body and a bodily lumen. For example, the systems, devices and methods can find use in laboratory and industrial settings for placing devices through one or more layers of material that may or may not find application to the human or animal body, e.g. for viewing between multiple layers of fabric, paintings, walls, etc. Some examples include construction or manufacturing, working with synthetic tissues, connecting or repairing polymeric sheets, animal studies, veterinary applications, and post-mortem activities.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A method of accessing an internal bodily cavity of patient, the bodily cavity defined by a cavity membrane, the patient having multiple tissue layers adjacent the bodily cavity including at least a skin layer and a fascia layer, the method comprising the steps of:
forming an opening through the skin layer and the fascia layer;
inserting an elongate medical device through the opening and between the fascia layer and the cavity membrane to form a tunnel communicating with the opening; and
utilizing a medical instrument within the tunnel.

2. The method of claim 1, further comprising the steps of grasping an edge of the fascia layer and lifting the edge of the fascia layer.

3. The method of claim 1, further comprising the step of separating the fascia layer from the cavity membrane.

4. The method of claim 3, wherein the step of separating the fascia layer includes pushing the elongate medical device distally to increase the size of the tunnel.

5. The method of claim 1, wherein the multiple tissue layers includes a second fascia layer, and wherein the step of inserting the elongate medical device includes passing the elongate medical device between the fascia layer and the second fascia layer, whereby the tunnel is formed between the fascia layer and the second fascia layer.

6. The method of claim 1, wherein the elongate medical device is one of a steerable wire guide and a steerable catheter.

7. The method of claim 6, wherein the medical instrument is an ultrasound detector formed as part of the flexible endoscope.

8. The method of claim 1, wherein the medical instrument is a plurality of magnets.

9. The method of claim 8, further comprising the step of providing an actuating magnet to attract the plurality of magnets to maintain a desired space between the cavity membrane and the fascia and skin layers.

10. The method of claim 1, wherein the medical instrument is one or more lighting devices.

11. The method of claim 1, wherein the medical instrument is a layer of shielding material.

12. The method of claim 1, wherein the tunnel extends laterally from the opening.

13. The method of claim 1, wherein the opening is formed posteriorly of a rib cage of the patient, and wherein the tunnel extends anteriorly from the opening to a position proximate the thoracic cavity.

14. A medical kit for accessing an internal bodily cavity of patient, the bodily cavity defined by a cavity membrane, the patient having multiple tissue layers adjacent the bodily cavity including at least a skin layer and a fascia layer, the medical kit comprising:
a cutting instrument for forming an opening in tissue layers;
a medical instrument capable of engaging and separating tissue layers for forming a tunnel entrance, the medical instrument sized for insertion into the opening;
a flexible elongate medical device sized for insertion into the opening and tunnel entrance and between tissue layers, the flexible elongate device having sufficient rigidity to further separate tissue layers through distal advancement of the elongate medical device through the tunnel entrance to increase the size of the tunnel.

15. The medical kit of claim 14, further comprising a second medical instrument sized for placement within the tunnel and between tissue layers.

16. The medical kit of claim 15, wherein the second medical instrument is a plurality of magnets.

17. The medical kit of claim 16, further comprising an actuating magnet to attract the plurality of magnets to maintain a desired space between the tissue layers.

18. The medical kit of claim 15, wherein the second medical instrument is one or more lighting devices.

19. The medical kit of claim 15, wherein the second medical instrument is a layer of shielding material.

20. The medical kit of claim 14, further comprising a laparoscopic port sized to be received within the opening.

21. A medical system for mechanically positioning tissue layers adjacent a bodily cavity of a patient, the medical system comprising:
a plurality of first magnets structured to be passed through an opening in the tissue layers and into a tunnel formed between the tissue layers;
a second magnet adapted to be positioned outside of the patient;
a frame structured for connection to the second magnet, the frame structured to be supported on at least one of an operating table, a floor, and the exterior of the patient; and
the second magnet attracting the plurality of first magnets with a collective attractive force sufficient to move and maintain the tissue layers away from the bodily cavity.

22. The medical system of claim 21, wherein the first magnets each have a smallest cross-sectional area less than about 15 square millimeters.
